(19) United States
(12) Patent Application Publication
(10) Pub. No.: US 2005/0267231 A1
(43) Pub. Date: Dec. 1, 2005

(54) Compositions and articles containing a crosslinked polymer matrix and an immobilized active liquid, as well as methods of making and using the same

(76) Inventor: Mark S. Pavlin, Savannah, GA (US)

Correspondence Address: INTERNATIONAL PAPER COMPANY, 6285 TRI-RIDGE BOULEVARD, LOVELAND, OH, 45140, US

(21) Appl. No.: 11/140,160

(22) Filed: May 27, 2005

Related U.S. Application Data
(60) Provisional application No. 60/574,759, filed on May 27, 2004.
(60) Provisional application No. 60/618,449, filed on Oct. 13, 2004.

(51) Int. Cl. A61L 2/00

(52) U.S. Cl. 523/102

(57) ABSTRACT

The invention relates to compositions and articles containing s crosslinked polymer matrix and an immobilized active liquid, as well as methods of making and using the same.

### BACKGROUND OF THE INVENTION

1. Field of the Invention

The invention relates to compositions and articles containing a polymer matrix and an immobilized active liquid therein, as well as methods of making and using the same.

2. Description of the Related Art

The curing and/or crosslinking of polymeric systems, for example epoxy systems, is described in textbooks and industrial handbooks such as “Handbook of epoxy resins” by Henry Lee and Kris Neville (McGraw Hill, 1967), “The Epoxy Formulators Manual” by the Society of Plastics Industry, Inc. (1984), and the Encyclopedia of Science and Technology (Kirk-Othmer, John Wiley & Sons, 1994). Until recently, curing such systems and others related thereto in a manner capable of immobilizing active liquids, such as those having and/or containing fragrance, has been very difficult, especially when durability and performance under a dynamic range of operation conditions are required from such systems.

For example, JP 032558899A requires the use of a solid powder system, while JP07145299 requires the use of a pre-formed urethane-containing epoxy resin cured in the absence of a polyamine and/or an active liquid containing a perfume. Further, the above-mentioned JP references refer specifically and only to fragranced articles, such as air fresheners. Because of this narrow goal to make such articles, the reaction and reaction products described therein fail to have a dynamic range of performance capabilities. Moreover, they fail to provide a product that is durable in the absence of a support. Therefore, a need arises for controllable reaction conditions that yield dynamic reaction products containing durable matrices capable of immobilizing any and/or all types of active liquids therein.

Compositions such as immobilized fragrance objects (e.g. IFOs), even more specifically air fresheners, are well known devices that release a fragrance into the air of a room of a house, area of a public building (e.g. a lavatory) or the interior of a car to render the air in that area more pleasing to the occupant. Only non-aqueous gels, for example, the thermoplastic polyamide-based products described in U.S. Pat. Nos. 6,111,655 and 6,503,577 as well as the thermoset poly(amide-acid)s of U.S. Pat. No. 5,780,527, are transparent solids that can be easily charged to a mold and thus formed into a visually attractive stable solid shape without the use of a support means. During preparation of such thermoplastic gels, the components must be heated to a temperature above the gelation temperature of the mixture, a process detrimental to the volatile and sometimes temperature sensitive active liquid such as a fragrance oil. During storage or use, these gels must not be exposed to low temperatures because they can turn unattractively cloudy. Further, high temperatures are inoperable because the gels flow at such temperatures and lose their shape or leak from their container. This drawback is serious for conditions requiring matrices to hold their shape at a dynamic range of temperatures, such as those car interior fresheners often exposed to temperatures in excess of 110° F. especially on hot summer days when the car is parked in direct sunlight. In addition, these polyamide gels are soft solids that are easily deformed if scraped, dropped, poked, or wiped. Thus, these conventional gels do not provide compositions and/or articles that are readily durable and capable of operating at a wide range of operating parameters.

### SUMMARY OF THE INVENTION

The present inventors have now discovered a more efficient solution to provide polymeric matrices containing immobilized active liquids that are extremely durable and dynamic so as to operate at a wide range of operating parameters.

One object of the present invention is to provide compositions and articles containing a polymeric matrix and an immobilized active liquid therein. The article may be an air freshener, a pharmaceutical distribution article, a neutraceutical distribution article, a bioceutical distribution article, an insect-resistant article, a mold-resistant article, a bacteria-resistant article, a pest-resistant article, an immobilized fragrance article, a decorative article, a biosensor and/or an analytical instrument. Methods of making and using such articles are also embodiments of the present invention.

Another object of the present invention is a reaction product of mixing a molecule having at least one functional group selected from an epoxy, an isocyanate, an anhydride, and an acrylate with a polyamine in the presence of an active liquid or water or mixtures thereof; as well as compositions and articles containing the same. Further, methods of making such the above-mentioned reaction products, compositions, and/or articles are a further object of the invention.

Another object of the present invention is a composition and/or article containing a cured polymeric matrix and an active liquid where the active liquid is immobilized within the cured polymeric matrix and the cured polymeric matrix is a reaction product of mixing a molecule having at least one functional group selected from an epoxy, an isocyanate, an anhydride, and an acrylate with a polyamine in the presence of an active liquid or water or mixtures thereof. One aspect of the invention optionally relates to embodiments when the cured polymeric matrix is a reaction product of mixing a molecule having at least one functional group selected from an epoxy, an isocyanate, an anhydride, and an acrylate with a polyamine in the presence of an active liquid or water or a reaction rate modifier (e.g. accelerator or reducer) or mixtures thereof. Further embodiments include method of making and using such compositions and articles.

Another object of the present invention is a composition and/or article containing a cured polymeric matrix and an active liquid where the active liquid is immobilized within the cured polymeric matrix and the cured polymeric matrix is a reaction product of mixing a molecule having at least one anhydride functional group and the molecule containing at least one anhydride functional group is not a maleic-based rubber. Further embodiments include method of making and using such compositions and articles.

Another object of the present invention is a composition and/or article containing a cured polymeric matrix and an active liquid where the active liquid is immobilized within the cured polymeric matrix and the cured polymeric matrix is a reaction product of mixing a molecule having at least one epoxy functional group and the polyamine is a non-aromatic polyamine. Further embodiments include method of making and using such compositions and articles.

Another object of the present invention is a composition and/or article containing a cured polymeric matrix and an active liquid where the active liquid is immobilized within the cured polymeric matrix and the cured polymeric matrix is a reaction product of mixing a liquid containing molecules having at least one functional group selected from an epoxy, an isocyanate, an anhydride, and an acrylate with a liquid containing a polyamine in the presence of an active liquid or water or mixtures thereof. Further embodiments include method of making and using such compositions and articles.

Another object of the present invention is a composition and/or article containing a cured polymeric matrix and an active liquid where the active liquid is immobilized within the cured polymeric matrix and the cured polymeric matrix is a reaction product of mixing a liquid containing molecules having at least one functional group selected from an epoxy, an isocyanate, an anhydride, and an acrylate with a polyamine in the presence of an active liquid or water or mixtures thereof; where the polyamine has an amine number of from 10 to 100 meq KOH/g and has a viscosity, measured at 150° C., of no greater than about 500 cP. Further embodiments include method of making and using such compositions and articles.

Another object of the present invention is a composition and/or article containing a cured polymeric matrix and an active liquid where the active liquid is immobilized within the cured polymeric matrix and the cured polymeric matrix is a reaction product of mixing a liquid containing molecules having at least one functional group selected from an epoxy, an isocyanate, an anhydride, and an acrylate with a polyamine in the presence of an active liquid or water or mixtures thereof; and where the polyamine is a liquid at room temperature. Further embodiments include method of making and using such compositions and articles.

Another object of the present invention is a composition and/or article containing a cured polymeric matrix and an active liquid where the active liquid is immobilized within the cured polymeric matrix and the cured polymeric matrix is a reaction product of mixing a liquid containing molecules having at least one epoxy functional group with a liquid containing a polyamine in the presence of an active liquid or water or mixtures thereof. Further embodiments include method of making and using such compositions and articles.

Another object of the present invention is a composition and/or article containing a cured polymeric matrix and an active liquid where the active liquid is immobilized within the cured polymeric matrix and the cured polymeric matrix is a reaction product of mixing a liquid containing molecules having at least one isocyanate functional group with a liquid containing a polyamine in the presence of an active liquid or water or mixtures thereof. Further embodiments include method of making and using such compositions and articles.

It is accordingly an additional object of the invention to provide a method for preparing transparent, flexible, and stable air care compositions by immobilizing active liquids with cured epoxy or isocyanate resins and air care products made thereby that overcome the disadvantages of the heretofore-known compositions and methods of this general type.

With the foregoing and other objects in view, there is provided in accordance with the invention, a method for preparing air care compositions and other useful articles. The method includes immobilizing active liquids such as fragrance oils or other active volatile or non-volatile liquids in a cured matrix, preferably crosslinked, selected from the reaction product of a polyamine and a liquid polyepoxy or liquid polyisocyanate material, the reaction being carried out in the presence of the active liquid. Products of this type may be prepared by: (1) blending the polyamine, the active liquid and any desired optional components including plasticizers, fillers, stabilizers, and colorants; (2) adding to this mixture the polyepoxy or polyisocyanate component optionally diluted with further amounts of plasticizers, fillers, stabilizers, and colorants; (3) optionally pouring the blend into a form, container, sheet or mold; (4) optionally covering or sealing the liquid mixture in the form, container, sheet or mold to prevent volatile components from evaporating; (5) optionally storing it until the mixture cures; and (6) optionally removing the immobilized liquid article from the form, container, sheet or mold and cutting it to shape or using it as made in the container.

One invention of the instant application is directed to providing a visually attractive solid air freshener, in particular a room, area, or car interior freshener, that is both transparent or nearly transparent (e.g. “frosted”) and robust. Robust means that the air freshener is packaged inexpensively and handled without being deformed. In particular, no special care is needed when the air freshener is taken out of its package or wrapper. Furthermore, the air freshener according to the invention is intended to resist changes in temperature and humidity, and exposure to light over the lifetime of its use or, with reasonable protection in a suitable package, over the lifetime of its storage and handling. The air care composition is to be essentially free of syneresis (also known as “sweating”) and that the matrix material of the product is to be effectively non-toxic and not cause skin irritation if handled outside of its storage wrapper. A further aspect of the invention is that the article components optionally may be made to be soluble or dispersible with water without losing any of the desired final properties (e.g. fragrance release, stability) of the article so that the water may optionally serve some useful purpose such as causing shrinkage to indicate end-of-life or introduction of a water-soluble active ingredient such as a salt.

In accordance with a further object of the invention, the active liquid may be and/or contain an aroma chemical and/or a bioactive material such as a deodorizer, sanitizer, insecticide, pesticide, repellant, or pheromone. For the latter four cases, the product would thus be a pest control device.

In accordance with a further object of the invention, the active liquid may be and/or contain a pharmaceutical, neutraceutical, and/or bioceutical. Thus, the invention relates, in part, to a patch for delivering a medical product to a human and/or animal patient.

In accordance with a further object of the invention, the active liquid may be and/or contain a biopolymer such as DNA, RNA, and/or protein, and/or a carbohydrate and/or a steroid. Of course all protected precursors of the same are envisioned by the present invention. Thus, the invention relates, in part, to a bioanalytical article and/or a biosensor for diagnostic purposes of patients, experimental subjects, and/or environmental factors.

In accordance with a further object of the invention, the air care composition preferably does not require the use of porous powders or fibers as a support for the fragrance oil.

In accordance with a further object of the invention, an inert solid material may be incorporated to make it more attractive. An example of such material may be, but is not limited to flakes, filings, glitter, foil, beads or chips of mica, metal, plastic, shell or glass or a natural material such as coffee grounds. A possible application for such a compound could be as a novelty coffee-mug coaster. Other solid objects may also be incorporated as desired, for example, a magnet to allow the cured article to cling to metal surfaces.

The invention of the instant application also encompasses a method. The method includes the steps of blending, preferably at or near room temperature, an active liquid such as a fragrance, a liquid polyepoxy compound or liquid polyisocyanate compound (herein referred to simply as an “epoxy” or an “isocyanate”), and a liquid or low melting polyamine (herein referred to as the “amine”); pouring the mixture into a mold or casting it into a sheet; optionally sealing the uncured or partially cured article in an impervious foil or film or container to prevent loss of the fragrance oil, and leaving the mixture undisturbed at room temperature or at an elevated temperature until it has cured. The resulting thermoset solid is useful as an air freshener or other device that releases the volatile component into the ambient air at a rate depending on the geometry of the product, the airflow around it under use conditions, and the loading of the fragrance.

When the composition and/or article according to one aspect of the invention contains a cured polymeric matrix and an active liquid where the active liquid is immobilized within the cured polymeric matrix and the cured polymeric matrix is a reaction product of mixing a molecule having at least one functional group selected from an epoxy and an isocyanate with a polyamine in the presence of an active liquid, there are Although the invention is exemplified and described herein as embodied in a method for preparing compositions and articles such as transparent, flexible, and stable air care compositions by immobilizing active liquids with cured matrices, it is nevertheless not intended to be limited to the details shown, since various modifications and structural changes may be made therein without departing from the spirit of the invention and within the scope and range of equivalents of the claims.

Matrix-immobilized active liquids are useful as air care or other devices because they releases the volatile fragrance or other active ingredient into the ambient environment at a rate depending on the geometry of the product, the airflow around it under use conditions, and the loading of the fragrance. The construction and method of operation of the invention, however, together with additional objects and advantages thereof will be best understood from the following description of specific embodiments when read in connection with the accompanying examples and claims.

### DESCRIPTION OF THE PREFERRED EMBODIMENTS

The present invention relates, in part, to a composition and/or article containing a polymeric matrix and an active liquid where the active liquid is immobilized within the polymeric matrix. The present invention also related to methods of immobilizing an active liquid within a polymeric matrix. Still further, the present invention relates to methods of making a composition and/or article containing a polymeric matrix and an active liquid where the active liquid is immobilized within the polymeric matrix. The active liquid may contain active ingredient or may be inherently active. The active liquid and/or active ingredients may be covalently and/or non-covalently attached to the polymeric matrix. The polymeric matrix may or may not be crosslinked.

The polymeric matrix is a reaction product of mixing a molecule having at least one functional group selected from the group consisting of an epoxy, an isocyanate, an anhydride, and an acrylate with a polyamine in the presence of an active liquid or water or mixtures thereof.

While the molecule having at least one epoxy functional group can be any epoxy, it is preferably a polyepoxy. Further, the epoxy-containing molecule is preferably in the form of a liquid. While examples of the epoxy containing molecule of the present invention can be found in “Handbook of epoxy resins” by Henry Lee and Kris Neville (McGraw Hill, 1967), “The Epoxy Formulators Manual” by the Society of Plastics Industry, Inc. (1984), and the Encyclopedia of Science and Technology (Kirk-Othmer, John Wiley & Sons, 1994), specific examples of liquid epoxy resins that may be of use in this invention are, but are not limited to, the diglycidyl ethers of bisphenol A and F (available as EPON®828 and EPON®8620 from Resolution Performance Products), hydrogenated glycidyl ether of bisphenol A (available as EPALLOY® 5000 and EPALLOY® 5001, products of CVE Specialty Chemicals), the diglycidyl ethers of butanediol, cyclohexane dimethanol, neopentyl glycol, dimer acid, and trimethylolpropane (all available from Resolution Performance Products in their HELOXY® Modifier product line).

The above-mentioned epoxy-containing molecules are merely representative and many additional epoxy-containing molecules are applicable in the present invention.

While the molecule having at least one isocyanate functional group can be any isocyanate, it is preferably a polyisocyanate. Further, the isocyanate-containing molecule is preferably in the form of a liquid. Specific examples of the isocyanate containing molecule of the present invention can be any aliphatic difunctional isocyanate material including well-known liquid diisocyanates such as isophorone diisocyanate and bis(4-isocyanato cyclohexyl) methane known as H-MDI. Preferred polyfunctional isocyanates of low volatility and thus reduced toxicity. Examples are those available from Bayer Corporation, Industrial Chemicals Division, under the trade names DESMONDUR including without limitation, the DESMONDUR N-series aliphatic isocyanates, especially DESMONDUR N-3300 and DESMONDUR N-3800, and the DESMONDUR Z-series, especially DESMONDUR Z4470.

The above-mentioned isocyanate-containing molecules are merely representative and many additional isocyanate-containing molecules are applicable in the present invention.

While the molecule having at least one anhydride functional group can be any anhydride, it is preferably a polyanhydride. Further, the anhydride-containing molecule is preferably in the form of a liquid. Still preferably, the anhydride-containing molecule is not a maleic-based rubber.

While the molecule having at least one acrylate functional group can be any acrylate, it is preferably a polyacrylate. Further, the acrylate-containing molecule is preferably in the form of a liquid.

The polyamine of the present invention may be any polyamine. Preferably, the polyamine is a liquid below 50° C., e.g. a low melting polyamine. More preferably, the polyamine is a liquid at or near room temperature. According to the invention, the polyamine has a melting or softening point below 50° C., including at most 50° C., 45° C., 40° C., 35° C., 30° C., 25° C., 20° C., and 15° C., including any and all ranges and subranges there between. Most preferably, the polyamine is a liquid and/or tacky and/or a semisolid at a temperature ranging from 50° C. to 10° C., including 50° C., 45° C., 40° C., 35° C., 30° C., 25° C., 20° C., 15° C., and 10° C., including any and all ranges and subranges there between. The polyamine is preferably aliphatic. Further the polyamine is preferably non-aromatic.

Preferred liquid polyamines are, but are not limited to, the following: polyether polyamines such as JEFFAMINE®D-230, T403, or XTJ-511, provided by Huntsman Corporation, and cycloaliphatic diamines such as isophorone diamine (IPDA), aminoethylpiperazine and 1,3-bis(aminomethyl) cyclohexane (1,3-BAC). Liquid polyamines may also be chosen from the polyamido-amine family, examples of which are the UNIREZ® series of amido-amine curing agents offered for sale by Arizona Chemical. These materials are known to impart adhesion and have lowered skin sensitivity. Polyamines may be mixtures of two or more polyamines blended to optimize viscosity, reaction rate and product performance.

Still further, a preferred polyamine of this invention has at least one of the following properties:
(a) has an amine number of from 1 to 100 meq KOH/g;
(b) dissolves in and is compatible with many active liquids;
(c) has a viscosity, measured at 150° C., of no greater than about 500 cP.

The amine number of the polyamine may be from 1 to 100 meq KOH/g. The amine number may be 1, 2, 5, 10, 15, 20, 25, 30, 35, 40, 45, 50, 55, 60, 65, 70, 75, 80, 85, 90, 95 and 100 meq KOH/g, including any and all ranges and subranges there between.

The viscosity, measured at 150° C., of the polyamine may be not greater than about 500 cP. The viscosity, measured at 150° C., of the polyamine may be not greater than about 475, 450, 425, 400, 375, 350, 325, 300, 275, 250, 225, 200, 175, 150, 125, and 100 cP, including any and all ranges and subranges there between.

The most preferred polyamine of this invention is, in addition to the above, a liquid at 25° C. and comprises polyether segments such that greater than 50% by weight of the polyamine is polyether derived from oligomerized ethylene oxide, propylene oxide, butylenes oxide or tetrahydrofuran or combinations of these. Examples of most preferred polyamines are polyalkyleneoxy polyamines supplied by, for example, Huntsman Corporation and BASF Corporation, specifically, Jeffamines D-2000 and T-5000 and the polyamide polyether block copolymers resulting from reaction of such polyalkyleneoxy polyamines with diacids as further described below and in the example section of this application.

Polyamide polyamines useful in the present invention can be made in one step by reacting a polyacid or mixture of polyacids with a poly(alkyleneoxy) polyamine admixed with piperazine and admixture further with another polyamine such as ethylene diamine, isophorone diamine, hexamethylene diamine, 2-methyl-1,5-pentane diamine, and the like.

Preferred is use of a majority amount of adipic acid, azeleic acid, or sebacic acid or other aliphatic diacid or its ester equivalent, determined as >50% of all acid equivalents present. Use of a majority amount of poly(alkyleneoxy) polyamine determined as >50% of all amine equivalents present, ensures that the resulting polyamide will have good solubility in a wide range of liquids including in certain cases, water. Also important to the proper controlled reactivity of the components is the amine number of the polyamide polyamide resin, which should be less than 100, as measured by titration with dilute alcoholic hydrochloric acid and expressed as mg KOH/g sample and preferably less than 80 mg KOH/g and most preferable less than 60 mg KOH/g.

Another preferred polyamide of this invention is the reaction product of diacids, the majority of which is polymerized fatty acid, also known as dimer acid (a material produced by Arizona Chemical Company, sold under the tradename “UNIDYME®” and by Cognis Corporation under the tradename “EMPOL®”) with a stoichiometric excess of polyamines, the majority of which are one or more poly(alkyleneoxy) polyamines chosen from the group of Huntsman Jeffamine® polyamines, including, for example, D-400, D-2000, T-403, and XTJ-500 and/or piperazine, such that, after the reaction is complete, the polyamide product is a liquid at room temperature, has an acid value of less than about 5 mg KOH/g and an amine value of from about 10 to about 60 mg KOH/g, and has a viscosity of less than 500 cP measured at 150° C. Most preferred is a polyamide polyamine that is a liquid at room temperature, has an acid value of less than 2 mg KOH/g and an amine value of 15-45 mg KOH/g, and has a viscosity of less than 500 cP at 150° C.

Co-diacids and co-diamines may be employed as long as the desired properties of the polyamide are obtained. Co-diacids may be, for example, adipic acid and similar linear aliphatic diacids, 1,4-cyclohexane diacid, and Westvaco 1550 C-21 diacid. Co-diamines may be, for example, ethylene diamine, 1,2-diaminocyclohexane, isophorone diamine, 1,3-bis(aminomethyl) cyclohexane, dimer diamine (Versamine®551), hexamethylene diamine, 2-methyl-1,5-pentane diamine, and similar linear, branched and cyclic aliphatic diamines.

Polyamines such as polyamide polyamines that are not liquids at room temperature, that are instead tacky solids at room temperature (e.g. low melting polyamines) are operable in the instant invention. Polyamines such as polyamide polyamines as described above that are non-tacky solids at room temperature and are compatible with the active liquids of this invention are operable in the present invention. Such preferable polyamides are obtained from the reaction of a major diacid portion of 1,4-cyclohexane dicarboxylic acid and a stoichiometric excess of polyamines, the majority of which are one or more poly(alkyleneoxy) polyamines chosen from the group of Huntsman Jeffamine® polyamines, including, for example, D400, D-2000, T-403, and XTJ-500 such that, after the reaction is complete, the polyamide product is a non-tacky solid at 25° C., has an acid value of less than 5 mg KOH/g and an amine value of from about 5 to about 60 mg KOH/g, and has a Ring & Ball softening point greater than 60° C. but less than 150° C. For these polyamides, dimer acid may optionally be used as a co-diacid along with other co-diacids such as those mentioned above. However, dimer acid is not essential, and in some instances, not preferable. Co-diamines likewise are optional components so long as their presence is not at a level sufficient to increase the softening point of the resin.

The polyamine of the present invention may also be those polyamines described in U.S. Pat. Nos. 6,870,011 and 6,399,713, as well as U.S. patent application Ser. No. 10/395,050, all such patents and patent applications are hereby incorporated, in their entirety, herein by reference.

The active liquid of the present invention may be any liquid that imparts upon the resultant composition and/or article a function. That is, the active liquid may be an organic volatile and/or non-volatile liquid or oil. Examples of such active liquids include fragrance oils.

The fragrance oil can be virtually any blend of the large number of synthetic aroma chemicals and natural oils known to the perfumer's art. Of some of the classes of chemicals useful in the instant invention are esters such as butyl acetate (present in banana oil), phenols such as methylsalicylate (present in oil of wintergreen), ethers such as 1,8-cineole (present in eucalyptus oil), alcohols such as geraniol (present in rose oil), aldehydes such as cinnamaldehyde (present in cinnamon oil) and ketones such as menthone (present in spearmint oil).

Specific examples of the many hundreds of commercially available fragrance oils useful in this invention are “Ocean” (N-123-03), “Country Wildflower” (N-122-03), “Spring Meadow” (N-124-03) and “Morning Rain” (Q-119-03) supplier by Continental Aromatics of Hawthorne, N.J.; “Evergreen” (#42441) and “Green Apple” (#50520) supplied by Belle Aire Fragrances of Mundelein, Ill.; “Cherry” (#124559), “Vanilla” (#122745) and “Mulberry” (#124561) supplied by Aromatic Fragrances and Flavors International of Marietta, Ga.; “Garnet” (#242926) supplied by International Fragrances Technology, Inc. of Canton, Ga.; and “Crisp Breeze”, “Tropical Fragrance”, “Oceanside Mist” supplied by Atlas Products of Tinley Park, Ill. A Table of examples is provided herewith below.

There are hundreds of commercial fragrance oils from scores of suppliers. The present invention is not limited to any particular fragrance, but a list provided below exemplifies the vast selection of oils that can be used to make immobilized oils of this invention and thus the dynamic operability of the polymeric matrix of present invention:

| | Supplier | Fragrance | Oil Code |
|---|---|---|---|
| Supplier | Location | Name | Number |
| Continental | Hawthorne, NJ | Ocean | N-123-03 |
| Aromatics | | Country | N-122-03 |
| | | Wildflower | |
| | | Spring | N-124-03 |
| | | Meadow | |
| | | Morning | Q-119-03 |
| | | Rain | |
| Orlandi, Inc. | Farmingdale, NY | Macintosh | 9466-16582 |
| Belle Aire | Mundelein, IL | Green | 50520 |
| | | Apple | |
| | | Yankee | 50522 |
| | | Home | |
| | | Evergreen | 42441 |
| Aromatic | Marietta, GA | Cherry | 124559 |
| Flavors and | | Vanilla | 122745 |
| Fragrances | | Downey | 127426 |
| | | Mulberry | 124561 |
| International | Canton, GA | Garnet | 242926 |
| Fragrances | | | |
| Technology | | | |
| Atlas | Tinley | Crisp | 4062184 |
| Products | Park, IL | Breeze | |
| | | Tropical | 4062182 |
| | | Fragrance | |
| | | Oceanside | 4062178 |
| | | Mist | |
| Wessel | Englewood | Orange | 11721 |
| Fragrances | Cliffs, NJ | Twist | |
| | | Linen | 15051 |
| | | Fresh | |
| | | Country | 6959 |
| | | Garden | |

The fragrance oil may be used in the compositions and/or articles of the present invention at levels from about 0.5% for very lightly scented objects to about 75% for objects poured into containers. A preferred fragrance oil level for air fresheners is about 10-55% and the most preferred use level is 20-40% by weight of the finished article not counting the weight of any embedded objects. The amount of fragrance oil may be 0.5, 1.0, 1.5, 2.0, 5.0, 10, 15, 20, 25, 30, 35, 40, 45, 50, 55, 60, 65, 70, and 75% for objects poured into containers, inclusive of all ranges and subranges there between. The amount of fragrance oil for air fresheners may be 10, 15, 20, 25, 30, 35, 40, 45, 50, and 55% by weight of the finished article not counting the weight of any embedded objects, inclusive of all ranges and subranges there between.

The active liquid may be water containing an active ingredient that is dissolved in water. The active liquid may be an inert organic containing an active ingredient that is dissolved in the organic liquid. Further, water and/or organic liquids may be used to dilute the active liquid or cause the active liquid to form an emulsion such as water-in-oil and/or oil-in-water emulsions.

Examples of active ingredient contained in the active liquid may be pharmaceuticals, neutraceuticals, and bioceuticals which are optionally combined with a biologically-acceptable carrier. Further, examples of the active ingredient contained in the active liquid may be biological molecule such as an amino acid, carbohydrate and/or steroid. Examples of the biological molecule may be a biopolymer or biocopolymer or chimera comprising DNA, RNA, oligonucleotides, modified DNA, modified RNA, proteins, polypeptides, and modified polypeptides.

In addition to preferred embodiments described above, additional embodiments are possible by changing the following parameters.
Plasticizers,
Compatibilizers,
Diluents,
Accelerators,
Tackifiers,
Fillers, and
Colorants.

Factors that can be used to affect epoxy products can be applied to the invention of the instant application as well. These materials may well impart benefits to the air fresheners of this invention.

Phthalates, benzoates, and lactate esters, alcohols, polyols, poly(propylene glycol)s and their alkyl ethers, and polyester polyols are examples of plasticizers that can be used in epoxy systems. These plasticizers may benefit the products of this invention by increasing flexibility.

Reactive diluents and inert diluents may also be used to lower initial blend viscosity.

Possible diluents include, but are not limited to, various mono- and diglycidyl ethers, glycols, and N-methylpyrollidinone.

These materials will function as compatibilizers, as will branched alcohols such as tridecyl alcohol, aromatic esters such as 2-ethylhexyl salicylate, and hydroxyl esters such as butyl lactate.

Phenols such as nonyl phenol and 2,4,6-tris(dimethylaminomethyl) phenol are examples of known accelerators of the epoxy-amine curing reaction. Therefore, they may benefit the system by shortening the time needed to cure the air fresheners of this present invention. Reaction accelerators may be any alcohol-containing molecule and/or water and/or mixtures thereof.

Dissolving certain resins in the epoxy or the diluent/plasticizer and adding them to the system may impart tack to the final product. These include rosin esters and polyterpenes sold by Arizona Chemical under the trade names SYLVATAC®, SYLVARES®, and SYLVALITE®.

The composition and/or article of the present invention may be made by contacting, mixing, blending, reacting and/or curing a molecule containing at least one functional group selected from an epoxy, isocyanate, anhydride, and acrylate with a polyamine in the presence of an active liquid. The resultant mixture may be preferably homogeneous.

Contacting, mixing, and blending of a molecule containing at least one functional group selected from an epoxy, isocyanate, anhydride, and acrylate with a polyamine in the presence of an active liquid may occur at a temperature ranging from 10-40° C. The polyamine, the active liquid, and the molecule containing at least one functional group selected from an epoxy, isocyanate, anhydride, and acrylate may be added in any order consecutively and/or at the same time. Of course, optional ingredients may also be added to the mixture in any order. In instances when the polyamine is a solid and/or tacky, it may optionally be mixed in a carrier, diluent, and/or the active liquid. The temperature may preferably be 10, 15, 20, 25, 30, 35, and 40° C., inclusive of all ranges and subranges there between.

Curing a pre-formed mixture may also be performed. However, curing may occur at the same time as the contacting, mixing, and/or blending step. Preferably the mixture will become a thermoset gel thereafter curing. Most preferably, the mixture will become a polymeric matrix containing an immobilized active liquid therein. Curing temperatures may be in the range from 10-110° C., preferably 10, 15, 20, 25, 30, 35, 40, 45, 50, 55, 60, 65, 70, 75, 80, 85, 90, 95, 100, 105 and 110° C., inclusive of all ranges and subranges there between.

Curing rate may be a function of at least six factors: temperature, epoxy:amine ratio, structure of the epoxy and the amine, accelerator concentration, composition of the fragrance oil, and concentration of the fragrance oil. Accordingly, the cure times can vary widely.

Mixing and/or curing can occur within a mold. For example, a low temperature procedure may include blending at room temperature, pouring into mold, sealing, and standing at room temperature. Such a procedure may take from 1-4 days. Another example is a pre-curing procedure which may include blending at room temperature, sealing tightly, heating to 70° C. for 30-90 minutes to obtain a partial cure but not gelling the composition, then pouring the resultant partial cure into a mold, letting it cool and stand at room temperature. Such a procedure may take from 0.5 to 2 days. Finally, another example is a high temperature procedure which may include blending at room temperature, pouring into a pouch or mold, sealing it tightly, and heating it to a temperature ranging from 60 to 100° C. Such a procedure may take from 1 to 5 hours.

One may not be limited to one of the above steps and may very well wish to combine different steps therein. In addition, the curing time may range from 1 hour to 4 days, more preferably 1, 2, 3, 4, 5, 6, 12, 24, 36, 48, 60, 72, 84, and 96 hours, inclusive of all ranges and subranges there between.

A preferred embodiment of the present invention includes blending a volatile oil, a liquid polyepoxy, and a liquid polyamine to form a mixture. Blending the components may occur at 10-40° C. However, the blending is performed so as not to cause a loss of the fragrance component. The temperature of blending may preferably be 10, 15, 20, 25, 30, 35, and 40° C., inclusive of all ranges and subranges there between.

When an epoxy-containing molecule is used, specific liquid polyamines recommended for use in this invention are, but are not limited to, the following: polyether polyamines such as JEFFAMINE®D-230, T-403, or XTJ-511, provided by Huntsman Corporation, and cycloaliphatic diamines such as isophorone diamine (IPDA), aminoethylpiperazine and 1,3-bis(aminomethyl) cyclohexane (1,3-BAC). Liquid polyamines may also be chosen from the polyamido-amine family, examples of which are the UNIREZ® series of amido-amine curing agents offered for sale by Arizona Chemical. These materials are known to impart adhesion and have lowered skin sensitivity. Polyamines may be mixtures of two or more polyamines blended to optimize viscosity, reaction rate and product performance.

When an epoxy-containing molecule is used, the temperature of curing can be room temperature, i.e. 25° C., but may be higher, depending on the temperature sensitivity of the active liquid component and its volatility. If the active liquid does not degrade readily and the curing is performed in a sealed mold, a preferred curing temperature is about 60° C. At this temperature, curing for a typical formulation takes place in about 3-6 hours, or less if accelerator is used.

An additional preferred embodiment of the present invention includes blending a volatile oil, a liquid polyisocyanate, and a liquid polyamine to form a mixture. Blending the components may occur at 10-40° C. However, the blending is performed so as not to cause a loss of the fragrance component. The temperature of blending may preferably be 10, 15, 20, 25, 30, 35, and 40° C., inclusive of all ranges and subranges there between. Preferably, a polyurea matrix is formed.

When an isocyanate-containing molecule is utilized, a preferred polyamine of this invention has the following properties:
(a) has an amine number of from 10 to 100 meq KOH/g;
(b) dissolves in and is compatible with many neat (as supplied) commercial fragrance oils;
(c) has a viscosity, measured at 150° C., of no greater than about 500 cP.

The reaction between the resin and the isocyanate is rapid at room temperature in the absence of a catalyst, so a catalyst may or may not be added. In some cases, a rate modifier may or may not be used to retard the reaction, allowing ample time for the ingredients to be blended and poured into a mold. Useful modifiers are for example the aldehydes and ketones normally present in common essential oils and fragrance oils. Others are those that are either bland in odor or enhance the odor of the active liquid. Examples of useful retardants are aromatic aldehydes and ketones such as benzaldehyde, acetophenone, cinnamic aldehyde, and methyl cinnamic aldehyde, terpenic aldehydes such as citral, citronellal, any of the ionone isomers, carvone, menthone, and camphor, C₄-C₁₈ aliphatic and cycloaliphatic aldehydes ketones such as isobutyraldehyde and methyl ethyl ketone, and the like. Further examples of a retarder are α,β unsaturated aromatic aldehydes.

It should be noted that while a catalyst such as a retarder described above may be preferred when an isocyanate-containing molecule is utilized according to the present invention, such catalysts (e.g. reaction rate retarder and/or accelerator) may be optionally utilized in all reactions according the present invention.

When an isocyanate-containing molecule is utilized, a preferred temperature of curing is room temperature, i.e. 25° C., but may be higher or lower, depending on the activity of the resin-hardener system and the temperature sensitivity and volatility of the active liquid. If the active liquid does not degrade readily and the curing is carried out in a sealed mold, a preferred curing temperature is about 50° C. At room temperature, curing for a typical formulation including carbonyl-functional retardant takes place in from about 20-200 minutes. The time may be 20, 25, 30, 35, 40, 45, 50, 55, 60, 65, 70, 75, 80, 85, 90, 95, 100, 110, 120, 130, 140, 150, 160, 170, 180, 190, and 200 minutes, including any and all ranges and subranges there between. In the absence of a retardant, typical curing times are about less than 1 minute to about 40 minutes. The time may be 1 second, 10 seconds, 30 seconds, 1 minute, 5 minutes, 10 minutes, 15 minutes, 20 minutes, 25 minutes, 30 minutes, 35 minutes, and 40 minutes, including any and all ranges and subranges there between.

In instances when invention relates, in part, to a composition and/or article according to one aspect of the invention contains a cured polymeric matrix and an active liquid where the active liquid is immobilized within the cured polymeric matrix and the cured polymeric matrix is a reaction product of mixing a molecule having at least one functional group selected from an epoxy and an isocyanate with a polyamine in the presence of an active liquid. Numerous advantages to immobilizing active liquids in this manner include:
Heat sensitive active liquids need not be subjected to heat; all product components can be blended at room temperature if desired.
The epoxies, isocyanates, and amines are compatible with a large variety of active liquids and in all proportions.
Epoxies, isocyanates, and amines having low odor, low color, and low toxicity are available.
Low and high loadings of active liquid in the product are possible.
All product components can be liquids that are easily blended together at room temperature with simple mixing equipment.
The cure reaction requires no external agent to trigger it, generates no volatile byproducts that might create bubbles, and occurs throughout the blend mass uniformly.
Little shrinkage of the blend occurs during curing.
The product is transparent if unfilled or un-emulsified with water, or if some incompatibility develops, slightly hazy.
The cured product has excellent durability, is insoluble in water, resists marring and attack by solvents, and will not melt when warmed.
Cured product does not adhere aggressively to most plastic packaging materials and so can be wrapped in materials that are not special release films.
Cured product tackiness can be adjusted so that the product can be removed from its package by hand and then stuck to a variety of vertical surfaces, e.g. a window.
The product can be readily colored with dyes and pigments and can be loaded, if desired, with typical Theological modifiers such as clays, polyamides, silicas, and cellulosics.
The fluid pre-cured liquid fills the mold so completely and easily that even fine details are captured as part of the finished product, for example, embossed logos, decorative designs and words.

When the composition and/or article according to another aspect of the invention contains a cured polymeric matrix and a active liquid where the active liquid is immobilized within the cured polymeric matrix and the cured polymeric matrix is a reaction product of mixing a molecule having at least one epoxy functional group with a polyamine in the presence of an active liquid, there are numerous advantages to immobilizing active liquids in this manner. For compositions and/or articles made with a matrix derived from an epoxy there are these advantages in addition to those described above:
Active liquid-epoxy-hardener blends cure slowly at room temperature allowing ample time for the blend to be degassed, blended with other components (as desired), handled, and then poured into molds before they become intractable.
A large variety of amines are available allowing final cured product properties to be controlled.

When the composition and/or article according to another aspect of the invention contains a cured polymeric matrix and a active liquid where the active liquid is immobilized within the cured polymeric matrix and the cured polymeric matrix is a reaction product of mixing a molecule having at least one isocyanate functional group with a polyamine in the presence of an active liquid, there are numerous advantages to immobilizing active liquid in this manner. For compositions and/or articles made with a matrix derived from an epoxy there are these advantages in addition to those described above:
The active liquids/polyamide/hardener blends cure rapidly at room temperature, often within 30 minutes.
Selection of the components allows for control of the cure time so that the blends can be degassed, blended with other components (as desired), handled, and then poured into molds before they become intractable;
The polyamide component, unlike other commercial amine hardeners, is a high molecular weight polymer having little or no odor, low color and low toxicity;

This process for immobilizing active liquids can be adjusted to overcome or avoid the following exemplified drawbacks:
Curing is exothermic but the heat generated in blends of this invention, in which the active liquids is a major component, is unnoticeable, especially when blends are allowed to cure in small molds with inherently good heat dissipation.
Epoxy curing is slow, often requiring 1-3 days at 25° C. but is desirably done in the mold after the product is sealed and packaged. Cure times may be significantly reduced by the use of accelerators and by heating the article to about 60° C.
Isocyanate curing in some cases is so rapid that the blend cannot be poured into a mold. This can be avoided by proper choice of the reactants, the blend temperature, and the fragrance oil formulation and amount.
Amines are somewhat toxic and require care in handling but are used in relatively low levels in the inventive compositions and become irreversibly incorporated into the crosslinked polymer matrix during the curing process. Only the meagerest traces of free amine are present in the cured product.
Isocyanates are somewhat toxic and require care in handling but are used in relatively low levels in the inventive compositions. Only the merest traces of free isocyanate are present in the cured product and can be eliminated over time by further reaction with alcohols in the active liquid or with added water.

Parameters that impact this invention are in the following categories:
Fragrance or other active liquid
Diluents or plasticizers
Amount of water present
Retardants, especially aldehydes
Fillers, glitter, crushed glass and the like
Icons (i.e. embedded solid objects)
Colorants (i.e. dyes and pigments)

Many of these are for the purpose of making the article more visually attractive. While not essential to the invention, these materials may well impart benefits to the articles of this invention such as modifying the release rate of the active ingredient contained therein.

The article of the present invention may be processed into any desired shape that is appealing to a potential consumer. Such shapes can be a 3-D shape formed in a mold or a flat shape stamp-cut from pre-formed thin sheets. Shapes may include those geometrical in nature, e.g. squares, circles, spheres, etc. Due to the immense number of 3-D shapes that may be formed the above-mentioned examples are not meant to be limiting to the articles of the present invention.

When the article of the present invention is an air freshener, it may be “active” and/or “passive”. Active air fresheners encompass relatively complex devices having moving parts such as heaters and fans to dispense concentrated or diluted aroma compounds or spray cans charged with aroma chemical, carrier liquid, and propellant. Active air fresheners require the occupant to dispense the material into the area to be treated. Passive air fresheners are available in many forms, but are in essences “fixed” liquid chemicals: a multi-component article including fragrance oil immobilized in and/or a solid support. The support material can be simple, e.g. a piece of cardboard, blotter paper, cotton, or other fibrous materials. The support material can be complex, e.g. an aqueous dispersion (gelatin) or a non-aqueous gel (gelled, e.g. by polyamide resin). Preferably, the air fresheners of the present invention are transparent, but can be opaque.

The articles of the present invention emit and/or release and/or adsorb and/or contain an active ingredient. Preferably article of the present invention release an active ingredient, such as a fragrance molecule. However, such articles may also

The present invention is explained in more detail with the aid of the following embodiment examples which are not intended to limit the scope of the present invention in any manner.

### EXAMPLES

### Example 1

Air freshener components (names and amounts listed below) including a small amount of green dye, were weighed into a glass vial and stirred together at ambient temperature by hand with a wooden stir stick. A portion of the mixture (8.0 g) was then poured into a flat, rectangular, 2.50 inch×3.25 inch uncoated polystyrene mold:
Epoxy Resin: EPALLOY® 5001, 10.00 g; 55.1%
Hardener: 1,3-BAC, 3.55 g; 19.6%
Fragrance Oil: Belle Aire “Evergreen”, 4.55 g; 25.1%
Dye: Green, 0.05 g; 0.3%.
   The next day the sample was firm, clear, tack-free, and flexible. It could be removed from the mold by hand with only a slight amount of sticking to the mold. Placed in a polyethylene “baggie” for storage at room temperature, it exhibited no syneresis, even after a number of weeks.

### Example 2

These air freshener components totaling 100 parts by weight were treated following the procedure of Example 1: EPALLOY®5001 (53.6 parts), 1,3-BAC (19.0 parts), Belle Aire “Evergreen” fragrance oil (25.1 parts), nonyl phenol (2.2 parts). The resulting article after curing at room temperature for one day was transparent, firm, flexible and tack-free.

### Example 3

These air freshener components totaling 100 parts by weight were treated following the procedure of Example 1: Cyclohexane dimethanol diglycidyl ether (22.8 parts), EPON®828 (22.8 parts), Huntsman T-403 polyamine (24.2 parts), Continental Aromatics “Country Meadow” fragrance oil (30.0 parts), plastic glitter 0.1 parts) and a trace of green dye. The resulting article after curing at room temperature for three days was transparent, firm, flexible, tack-free and exhibited ability to cling lightly to a flat vertical glass surface from which it could be easily removed and re-applied without marring the surface.

### Example 4

A polyamide polyamine was prepared by charging adipic acid (20.0 g, 274 meq acid), Jeffamine®T403 polyamine (20 g, 132 meq amine) and Huntsman XTJ-500 (80 g, 254 meq. amine) to a 250 mL glass flask equipped with a stirrer and heating this charge to 210-220° C. under a stream of dry nitrogen. After holding this mixture under these conditions for 5 hours, the reaction mixture was discharged to a container. The product was a clear, viscous, nearly water-white liquid having an acid number of 1.4, an amine number of 42.2, and a Brookfield viscosity at 150° C. of 340 cP. A portion of this product (11.63 g) was dissolved in water (27.5 g) and then blended with a polyethyleneglycol diglycidyl ether (EEW of 195; 3.40 g). To a portion of this mixture (20.0 g) in a small plastic jar with a screw cap was then added fragrance oil (“Sunshine Fruits”, Firmenich fragrance oil #190196) and a few drops of Tween 80 surfactant, forming a milky emulsion which, after being capped and allowed to stand, gelled to an immobile firm homogeneous white solid that emitted the fragrance gradually after being un-capped.

### Example 5

To a commercial resealable polyethylene “baggie” was added components totaling 100 parts by weight: cyclohexane dimethanol diglycidyl ether (13.9 parts), EPON®826 (13.9 parts), Arizona proprietary liquid triethylenetetraamine-based amido-amine #X54-327-004 (amine number of 349, acid number of 0.8, 22.2 parts), Atlas “Crisp Breeze” fragrance oil (50.0 parts), and a trace of blue dye. The “baggie” was massaged to blend the components for a few minutes, the air bubbles pressed out and the fluid mixture then stored lying flat at room temperature for one week. At that time the material was cured to the point of being immobile, transparent, and flexible.

### Example 6

To a glass beaker containing a magnetic stir bar was charged Huntsman Surfonic®L24-5, a liquid ethoxylated alcohol surfactant (12.0 g), Atlas Products “Crisp Breeze” fragrance oil (8.0 g), Huntsman T-403 polyamine (8.4 g), FD&C #3 blue-green dye (0.4 g) and HELOXY®48 epoxy resin (14.0 g). This mixture was heated to 58° C. for about 3 hours with stirring to nearly cure it and then poured into a cylindrical mold and allowed to cool. After the material stood about three days at room temperature it was removed from the mold as a slightly rubbery, firm solid.

### Example 7

These air freshener components totaling 100 parts by weight were blended at room temperature: cyclohexane dimethanol diglycidyl ether (25.3 parts), EPON®828 (17.2 parts), Arizona proprietary polyamido-amine hardener #X54-327-004 (34.5 parts), Continental Aromatics “Ocean” fragrance oil (23.0 parts), and a trace of green dye. This blend was held for about 45 minutes at about 67° C., at which time it was allowed to cool to room temperature. It was, at this stage, quite viscous, but could still be poured and stirred. To this partially cured intermediate was added with gentle distribution through the mass approximately two dozen ¼″ colored foil hearts. The resulting article after curing at room temperature for three days was firm, flexible, and tack-free with the foil hearts clearly visible suspended uniformity inside it.

### Example 8

These components totaling 100 parts by weight were treated following the procedure of Example 1: poly(propylene glycol) diglycidyl ether (13.0 parts), EPON®828 (22.0 parts), Arizona UNI-REZ®2801 amido-amine (14.0 parts), “Vanilla” fragrance oil from Aromatic Flavors and Fragrances, dipropyleneglycol benzoate (19.5 parts) and commercial ground coffee (29.5 parts). The resulting article after curing was firm, slightly flexible, non-tacky. The coffee grounds were uniformity distributed and gave the article a rich brown, opaque apperance, smooth at the bottom where the mold was smooth and rough on top where the grounds were allowed to settle freely.

In the following examples, abbreviations are as follows:
CHDA is 1, 4 cyclohexane dicarboxylic acid from Eastman Chemical;
Empol is EMPOL® 1008 dimer acid supplied by Cognis Corporation;
Unidyme is UNIDYME®18 dimer acid supplied by Arizona Chemical Company;
T-403 is JAFFAMINE®T-403 poly(alkyleneoxy) diamine supplied by Huntsman Corporation;
D-400 is JEFFAMINE®D-400 poly(alkyleneoxy) diamine also from Huntsman;
D-2000 is JEFFAMINE®T-2000 poly(alkyleneoxy) diamine also from Huntsman;
V-551 is VERSAMINE®551 dimer diamine supplied by Cognis Corporation;
N-3300 is DESMONDUR N-3300, Bayer Corporation, Industrial Chemicals Division;
N-3800 is DESMONDUR N-3800, also from Bayer;
Z-4470 is DESMONDUR Z4470, also from Bayer.

### EXAMPLE 9

A polyamide polyamine was prepared by charging EMPOL®1008 polymerized fatty acid (63.0 g, 219 meq acid), Jeffamine®T403 polyamine (18 g, 118 meq amine) and Jeffamine®D-400 (45 g, 205 meq. amine) to a 250 mL glass flask equipped with a stirrer and heating this charge to 210-220° C. under a stream of dry nitrogen. After holding this mixture under these conditions for 5 hours, the reaction mixture was discharged to a container. The product was a clear, viscous, nearly water-white liquid having an acid number of 0.3, an amine number of 41.8, a weight average molecular weight of 2,270, and a Brookfield viscosity at 150° C. of 204 cP.

A solution was prepared by warming 10.0 g of this polyamide polyamine with 5.0 g Finsolv™TN benzoate ester plasticizer and 10.0 g fragrance oil (“Linen Fresh”, Wessel Fragrances), cooled to room temperature and blended thoroughly with a mixture of DESMONDUR Z4470 and 5.1 g additional fragrance oil. To the composition was then added a small amount of red dye and red glitter. A few minutes later about 25 g of this final formulation was poured into a flat, circular rose-shaped silicone rubber mold and the remainder retained in a jar. A total of 33 minutes after the component were blended, the retained material was set to an immobile gel. After standing at room temperature for 16 hours, the immobilized fragrance oil article was removed from the mold. It did not adhere to the mold, was non-tacky, had the exact flower shape of the mold, exhibited a uniform color and distribution of glitter, and could be handled without breaking up. It also exhibited excellent cling to a variety of vertical surfaces including glass and plastic film.

### Examples 10-15

Polyamide polyamines were prepared according to the procedure of Example 9 by charging acids and amines of the types listed in the TABLE A (below) in the weight percentages indicated to a reactor and heating the charge to 200-220° C. under a stream of dry nitrogen for about 4-5 hours and discharging the product. Products properties were then measured and are also recorded in TABLE A.

Immobilized fragrance oils were prepared by warming a mixture of 2.0 grams polyamide polyamine of the example and 2.0 grams fragrance oil to about 55° C. and then blending the warm

TABLE A
Polyamide Polyamines Of Examples 10-15

| | EXAMPLE NUMBER | | | | | |
|---|---|---|---|---|---|---|
| | Example 10 | Example 11 | Example 12 | Example 13 | Example 14 | Example 15 |
| COMPONENTS | | | | | | |
| DiAcid | Adipic Acid | Empol | Empol | Empol | CHDA | Unidyme |
| Diamine | T-5000 | T-403 | T-403 | T-403 | T-403 | D-2000 |
| Co-DiAmine | — | D-400 | D-400 | XTJ-500 | D-400 | Piperazine |
| Third Diamine | — | D-2000 | D-2000 | — | D-2000 | |
| COMPONENTS (Wt. %) | | | | | | |
| DiAcid | 2.0% | 41.2% | 30.8% | 43.3% | 18.7% | 82.3% |
| Diamine | 98.0% | 9.6% | 4.2% | 12.6% | 17.8% | 2.1% |
| Co-DiAmine | 0.0% | 24.6% | 16.7% | 44.1% | 35.5% | 15.6% |
| Third Diamine | 0.0% | 24.6% | 48.3% | 0.0% | 28.0% | 0.0% |
| PRODUCT PROPERTIES | | | | | | |
| Neutralization | 194.4% | 139.5% | 141.5% | 148.2% | 141.1% | 131.7% |
| Acid Number | 0.4 | 0.5 | 0.4 | 0.4 | 1.4 | 0.6 |
| Amine Number | 12.2 | 27.1 | 22.6 | 42.4 | 44.6 | 14.1 |
| Color | Pale yellow | Pale yellow | Pale yellow | Off-White | Pale yellow | Amber |
| Softening Point (R&B, ° C.) | Liquid | Liquid | Liquid | Liquid | 128 | Liquid |
| Viscosity At 150° C. | 770 | 391 | 141 | 190 | 290 | 481 |
| Wt. Aver. Mol. Wt. | 6,150 | 2,150 | 17,780 | 5,650 | 1,720 | 33,760 |

mixture by hand with a stir stick. Test fragrances were: “Ocean” (Continental Aromatics), “Linen Fresh” (Wessel Fragrances), and “Cherry” (Aromatic Flavors and Fragrances). After blending, one equivalent of isocyanate hardener dissolved in an equal weight of oil was added with manual stirring, a stopwatch was started, and the mixture monitored for its consistency. When the mixture no longer could flow under its own weight, the time (in minutes) was noted as the “gel time”. TABLE B shows that all of these polyamide polyamines were effective in immobilizing the target oils when cured with polyisocyanates. Gel times were short but not so short as to preclude the preparation of useful articles and followed the consistant pattern:

Ocean<Linen Fresh<<Cherry

TABLE B
Gel Times of The Formulations of Examples 10-15
(Minutes at 50 Wt % Polyurea)

| GEL COMPONENTS | | POLYAMIDE POLYAMINE OF EXAMPLE | | | | | | |
|---|---|---|---|---|---|---|---|---|
| Fragrance Oil Type | Hardener | No. 9 | No. 10 | No. 11 | No. 12 | No. 13 | No. 14 | No. 15 |
| Ocean | N-3300 | 6.5 | 15 | 10 | 40 | 8.5 | 10 | 73 |
| Linen Fresh | N-3300 | 9 | 24 | 13 | 55 | 10 | 13 | 76 |
| Linen Fresh | Z4470 | 33* | 44 | 22 | nd | nd | nd | nd |
| Cherry | N-3300 | 75 | 170 | 95 | 335 | 87 | nd | nd |

*40% polyurea- see Example 9 for conditions

### Examples 16-20

Polyamide polyamines were prepared according to the procedure of Ex. 9 by charging acids and amines of the types listed in the TABLE C in the weight percentages indicated to a reactor and heating the charge to 200-220° C. under a stream of dry nitrogen for about 5 hours and discharging the product. Products properties were then measured and are also recorded in TABLE C.

Immobilized fragrance oils were prepared by warming a mixture of 2.0 grams polyamide polyamine of the example and 2.0 grams fragrance oil to about 55° C. and then blending the warm mixture by hand with a stir stick. Test fragrances were: Oceanside Mist, Tropical (Atlas Products), Spring Meadow, Country Wildflower, Ocean (Continental Aromatics), Linen Fresh (Wessel Fragrances), Yankee Home (Belle Aire), Mulberry and Cherry (Aromatic Flavors and Fragrances). After blending, one equivalent of isocyanate hardener dissolved in an equal weight of oil was added with manual stirring, a stopwatch was started, and the mixture monitored for its consis-tency. When the mixture no longer could flow under its own weight, the time (in minutes) was noted as the “gel time”. TABLE D shows that all of these polyamide polyamines were effective in immobilizing the target oils when cured with polyisocyanates. Gel times were short but not so short as to preclude the preparation of useful articles and followed the consistant pattern:

Spring Meadow<Ocean<Tropical<Linen Fresh<Yankee Home<Mulberry<Cherry

TABLE C
Polyamide Polyamines Of Examples 8-20

| | EXAMPLE | | | | |
|---|---|---|---|---|---|
| | No. 16 | No. 17 | No. 18 | No. 19 | No. 20 |
| COMPONENTS | | | | | |
| DiAcid | Empol | Adipic Acid | Adipic Acid | Empol 1008 | Unidyme |
| Triamine | T403 | T403 | T403 | — | — |
| Diamine | D-400 | XTJ-500 | D-400 | D-400 | V-551 |
| Third Amine | D-2000 | — | D-2000 | D-2000 | — |
| WEIGHT % | | | | | |
| DiAcid | 30.6% | 18.2% | 15.2% | 36.7% | 41.7% |
| Triamine | 5.0% | 9.1% | 7.6% | — | |
| Diamine | 16.5% | 72.7% | 38.6% | 22.9% | 58.3% |
| Third Amine | 47.9% | — | 38.6% | 40.4% | — |
| PROPERTIES | | | | | |
| Acid Number | 0.6 | 2.2 | 0.7 | 0.7 | 1.1 |
| Amine Number | 27.0 | 28.9 | 29.9 | 13.1 | 33.2 |
| Color | Colorless | Colorless | Colorless | Colorless | Amber |
| Viscosity [cP at 150° C.] | 106 | 393 | 198 | 1340 | 656 |
| Weight Aver. MW | 26380 | 12230 | 13490 | 31550 | 13180 |

TABLE D
Gel Times of The Formulations of Examples 16-20
(Hardener is N3300. minutes at 50 wt % polyurea)

| | Polyamide Polyamine of Example | | | | |
|---|---|---|---|---|---|
| Fragrance Oil Type | No. 16 | No. 17 | No. 18 | No. 19 | No. 20 |
| Oceanside Mist | Nd | nd | nd | 41 | nd |
| Spring Meadow | Nd | nd | nd | 42 | nd |
| Country Wildflower | Nd | nd | nd | 75 | nd |
| Ocean | 32 | 14 | 18 | >180 | 4 |
| Tropical | 38 | nd | 29 | >180 | nd |
| Linen Fresh | 40 | 20 | 32 | 225 | 13 |
| Yankee Home | 80 | 27 | 51 | >180 | nd |
| Mulberry | 315 | 185 | 250 | nd | nd |
| Cherry | >420 | 360 | >300 | >180 | 240 |

As used throughout, ranges are used as a short hand for describing each and every value that is within the range, including all subranges therein.

Numerous modifications and variations on the present invention are possible in light of the above teachings. It is, therefore, to be understood that within the scope of the accompanying claims, the invention may be practiced otherwise than as specifically described herein.

All of the references, as well as their cited references, cited herein are hereby incorporated by reference with respect to relative portions related to the subject matter of the present invention and all of its embodiments.

1) A composition comprising:
a cured polymeric matrix; and
an active liquid,
wherein said active liquid is immobilized within the cured polymeric matrix; and
the cured polymeric matrix is a reaction product of mixing a molecule having at least one functional group selected from the group consisting of an epoxy, an isocyanate, an anhydride, and an acrylate with a polyamine in the presence of an active liquid or water or mixtures thereof.

2) The composition according to claim 1, wherein the cured polymeric matrix is a reaction product of mixing a molecule having at least one functional group selected from the group consisting of an epoxy, an isocyanate, an anhydride, and an acrylate with a polyamine in the presence of an active liquid or water or a reaction accelerator or mixtures thereof.

3) The composition according to claim 1, wherein the cured polymeric matrix is a reaction product of mixing a molecule having at least one functional group selected from the group consisting of an epoxy, an isocyanate, an anhydride, and an acrylate with a polyamine in the presence of a active liquid or water or a reaction retarder or mixtures thereof.

4) The composition according to claim 1, wherein the cured polymeric matrix is a reaction product of mixing a molecule having at least one anhydride functional group and the molecule containing at least one anhydride functional group is not a maleic-based rubber.

5) the composition according to claim 1, wherein the cured polymeric matrix is a reaction product of mixing a molecule having at least one epoxy functional group and the polyamine is a non-aromatic polyamine.

6) The composition according to claim 1, wherein the cured polymeric matrix is a reaction product of mixing a liquid comprising molecules having at least one functional group selected from the group consisting of an epoxy, an isocyanate, an anhydride, and an acrylate with a liquid comprising a polyamine in the presence of an active liquid or water or mixtures thereof.

7) The composition according to claim 1, wherein the cured polymeric matrix is a reaction product of mixing a liquid comprising molecules having at least one functional group selected from the group consisting of an epoxy, an isocyanate, an anhydride, and an acrylate with a polyamine in the presence of an active liquid or water or mixtures thereof; wherein the polyamine has an amine number of from 10 to 100 meq KOH/g and has a viscosity, measured at 150° C., of no greater than about 500 cP.

8) The composition according to claim 1, wherein the cured polymeric matrix is a reaction product of mixing a liquid comprising molecules having at least one functional group selected from the group consisting of an epoxy, an isocyanate, an anhydride, and an acrylate with a polyamine in the presence of an active liquid or water or mixtures thereof; and wherein the polyamine is a liquid at room temperature.

9) The composition according to claim 1, wherein the cured polymeric matrix is a reaction product of mixing a liquid comprising molecules having at least one epoxy functional group with a liquid comprising a polyamine in the presence of an active liquid or water or mixtures thereof.

10) The composition according to claim 1, wherein the cured polymeric matrix is a reaction product of mixing a liquid comprising molecules having at least one isocyanate functional group with a liquid comprising a polyamine in the presence of an active liquid or water or mixtures thereof.

11) An article comprising
the composition according to claim 1.

12) The article according to claim 11, wherein said article is at least one article selected from the group selected from the group consisting of an air freshener, a pharmaceutical distribution article, a neutraceutical distribution article, a bioceutical distribution article, an insect-resistant article, a mold-resistant article, a bacteria-resistant article, a pest-resistant article, an immobilized fragrance article, a decorative article and a biodetection article.

13) A method of making the composition, comprising
mixing a molecule having at least one functional group selected from the group consisting of an epoxy, an isocyanate, an anhydride, and an acrylate with a polyamine in the presence of an active liquid to form a mixture.

14) The method according to claim 13, further comprising
curing the mixture.

15) The method according to claim 13, further comprising
mixing a reaction accelerator to the mixture.

16) The method according to claim 13, further comprising
mixing a reaction retarder to the mixture.

17) The method according to claim 13, wherein the polyamine is non-aromatic.

18) The method according to claim 13, wherein the molecule having at least one functional group selected from the group consisting of an epoxy, an isocyanate, an anhydride, and an acrylate is not a maleic-based rubber.

19) The method according to claim 13, wherein the polyamine has an amine number of from 10 to 100 meq KOH/g and has a viscosity, measured at 150° C., of no greater than about 500 cP.

20) The method according to claim 13, wherein the polyamine is mixed with a molecule having at least one epoxy functional group.

21) The method according to claim 13, wherein the polyamine is mixed with a molecule having at least one isocyanate functional group.
